(19) United States
(12) Patent Application Publication
(10) Pub. No.: US 2002/0052045 A1
(43) Pub. Date: May 2, 2002

(54) Method for kernel culture

(75) Inventor: Adriana Tomas, Des Moines, IA (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Pioneer Hi-Bred International, Inc.

(21) Appl. No.: 09/742,722

(22) Filed: Dec. 21, 2000

Related U.S. Application Data
(60) Provisional application No. 60/173,200, filed on Dec. 27, 1999.

(51) Int. Cl. C12N 5/00

(52) U.S. Cl. 435/424

(57) ABSTRACT

A method for kernel culture is described. The method provides certain advantages, including use of less culture medium, decreased abortion rates, and less plant-to-plant variability.

### CROSS REFERENCE

This application claims benefit of U.S. Provisional Application No. 60/173,200, filed Dec. 27, 1999.

### FIELD OF THE INVENTION

The present invention relates generally to laboratory procedures useful in plant biology. More specifically, the present invention relates to a method for kernel culture.

### BACKGROUND OF THE INVENTION

It has been known since the late 1970s that the process of fertilization and subsequent kernel (caryopsis) development of maize (Zea mays L.) can occur in vitro. See, e.g., Gengenbach, 1977a and 1977b. Earlier work had facilitated the in vitro pollentation and fertilization of various dicot plant types, but Gengenbach (1977a) reported that until 1977 there were no reports of successful kernel culture in any monocot, including maize.

The methods of Gengenbach (1977a and b) allowed researchers to study, in greater detail, kernel development (Jones et al., 1981), nutrient uptake into seeds (Shimamoto and Nelson, 1981), protein synthesis in kernels (Cully et al., 1984), metabolic processes in kernels (Misra and Oaks, 1985; Ly Zhik et al., 1985), aspects of nitrogen uptake and utilization (Singeltary and Below, 1989; Singeltary et al., 1990; Czyzewicz and Below, 1994), and other aspects of kernel/seed physiology and biochemistry (Duke and Doehlerf, 1996; Doehlert et al., 1997). Although the currently practiced kernel culture methods have proven to be quite useful, certain aspects of those methods remain problematic.

First, the current kernel culture methods available in the art use a high (typically 1:6) kernel-to-cob ratio. This can result in an unfavorably high inherent plant-to-plant variability. Second, the currently accepted kernel culture methods require relatively large volumes of culture medium on a per kernel basis. Although abortion ratios have not been reported in the literature, if a 0% abortion rate is assumed, Culley et al. (1984), plating on agar medium, used as average of 8 ml of medium per kernel. Singletary et al (1989 and 1990) used liquid medium and a wire/paper support platform in 23-31 ml/kernel.

The present invention addresses these issues by providing a method for kernel culture in which a kernel-to-cob ratio as low as about 1:2 can be employed. A further advantage of the present invention is that it allows the use of much reduced volumes of culture medium; on a per kernel basis the volume can be as low as 0.9 ml, with low abortion rates.

### SUMMARY OF THE INVENTION

The present invention therefore provides a method for kernel culture comprising the sectioning and sub-sectioning of harvested ears of a plant, such as a member of the genus Zea, so that the kernel-to-cob ratio is less than 1:6; preferably in a range of 1:2 to 1:5, and most preferably about 1:2. These subsections are cultured on florist foam in a suitable container and in a culture medium suitable for the type of experiments to be carried out on the cultured kernels.

### DETAILED DESCRIPTION

### EXAMPLE 1

Ear shoots are kept bagged until plants are pollinated, 2-3 days after silk emergence. Ears are harvested four days after pollination (DAP), placed on cide, and processed within four hours of harvesting essentially following the method of Gengenbach (1997) except for the size of the kernel block. In the field, protruding silks are cut off and the most outer husks removed. Once in the laboratory, all steps are conducted in a laminar flow hood using aseptic conditions. Holding ears by the shank, each ear is sprayed with alcohol and flamed before removing the outer husks. When only the most inner layer of husks was left, the ear is again lightly sprayed with alcohol and flamed.

Sections of kernels, two kernels wide and with cob tissue at least 3 mm deep, are removed from the middle third of the ear. The wedges are further sectioned to eight-kernel blocks and the two kernels at each end of the cob piece carefully removed to leave four developing kernels per block. Care is taken during excision to prevent drying out of kernels and cob tissue.

Culture trays consist of disposable tissue culture Phytatrays (Sigma P5929, St. Louis, Mo.) each with a sterile 1.5 cm deep brick of florist foam (Wet Foam Block, StyroFab, Waxahachie, Tex.). The tray lid—used as base—is filled with 75-80 mls of culture medium, enough to completely soak the foam and leave 2 mls of free medium. Kernel blocks are pressed onto the wet foam brick until the kernels are leveled with the surface of the brick. On average, each tray contains 12-15 four-kernel blocks. With a hot dissecting needle, four small holes are made high on the sides of the tray lids to allow for some gas exchange. Foam bricks and liquid culture medium are sterilized by autoclaving for 30 min. The culture medium used is a modification of that used by Culley et al. (1984) and contains 4.33 g Murashige Skoog Salts, 2 g L-asparagine monohydrate, 400 mg thiamine hydrocholoride, and 10 mg streptomycin sulfate per liter, brought to pH 5.8 with 4N HCL before autoclaving for 35 min. The carbon is added before sterilization and consists of 80 g/l glucose or sucrose.

Cultures are incubated at 24° C. in the dark, and inspected every other day for possible contamination. After 7 days in culture, kernel blocks are transferred to fresh culture trays containing labeled medium and incubated for an additional 16-20 days with no changes of medium. Overall, kernels are harvested between 27-31 DAP. At harvest, kernels are classified as small, large, cracked or popped, and aborted pooled accordingly, and weighed before freezing at −80° C.

Results from Experiment AT234703

Abortion rates were measured within and after the first two weeks in culture. Assuming all ovules plated had been fertilized, cultures grown in glucose medium sustained an average 15% early abortion and a further 21% late abortion. Of all kernels plated on glucose, 66% developed into unbroken kernels while 11% had broken pericarps and exposed endosperms (‘popped’). Cultures grown with sucrose/acetate as the carbon source sustained an average 9% early abortion and a further 3% late abortion. Of all plated kernels, 72% were classified as whole kernels and 17% as popped. Large kernels weighed an average of 221 mg in glucose and 187 mg in sucrose. Small kernels weighed an average of 136 mg in glucose and 133 mg in sucrose.

The present kernel culture method presents two advantages over previously used designs. In our protocol, a 1:2 kernel/cob ratio was sufficient to ensure proper kernel growth. This is in sharp contrast to the 1:6 kernel/cob ratio used in most studies (Gengebach, 1977; Culley et a., 1984; Singletary et al., 1989,1990). Since many more kernels can be obtained from one ear, the inherent plant to plant variability is reduced.

A second advantage relates to the volume of culture medium needed per kernel cultured. In previously reported studies, large volumes of medium were used per kernel cultured. Unfortunately, abortion ratios were not reported. Assuming 0% abortion (not a likely occurrence), Culley et al. (1984) plating on agar media, used an average of 8 mls of medium per kernel; Singletary et al. (1989,1990), using liquid medium and a wire/paper platform, employed 23-31 mls of medium per kernel. Using florist foam as substrate, and taking into account abortion rates of 10-23%, only 0.93-2.16 mls of medium are used per harvested kernel. The present method makes much more efficient use of expensive labeled material than the previously reported systems.

### EXAMPLE 2

In this example, auxin biosynthesis was analyzed in maize (Zea mays) kernels in the culture system of the present invention.

### MATERIALS AND METHODS

Materials

[U-¹³C₆]glucose was purchased from Isotec, Miamisburg, Ohio. L-[ring-²H₅]Trp, [3-¹³C]indole and [¹³C]formaldehyde were purchased from Promochem GMBH, Wesel, Germany.

Synthesis of [3,3′-¹³C₂]tryptophan

1. 1H-[3-¹³C]indol-3-yl[¹³C]methyl)-N,N-dimethylamine (a): glacial acetic acid (700 μl) was mixed with 700 μl of an 20% (w/v) aqueous solution of ¹³C-formaldehyde and 450 μl of an aqueous solution of 50% (w/v) dimethylamine HCl. The mixture was cooled to 0° C. 3-¹³C-indole (500 mg ,4.24 mmol) were added with stirring. After 3 h, 10 ml of 2M NaOH were added to the clear solution. A precipitate of colorless crystals of (a) was formed immediately. The mixture was extracted twice with 20 ml ethyl acetate. The organic layer was dried (Na₂SO₄) and evaporated. Yield: 723 mg (4.11 mmol, 97%).

2. -acetylamino-2-(1H-[3-¹³C]indol-3-yl)[3′-¹³C]methyl)propionic acid diethyl ester (b): 95 mg (4.13 mmol) of sodium were dissolved in 9 ml of absolute ethanol. 720 mg (4.09 mmol) of (a) were added to the sodium ethanolate solution. Then 900 mg of acetamino diethyl malonate and finally 1020 mg of dimethylsulfate were added. The solution was stirred for 12 h. Then 50 ml of H₂O were added. The mixture was extracted twice with each 20 ml of ethyl acetate, dried (NaSO₄) and the solvent was removed. Yield: 1.388 g (97%).

3. 2-acetylamino-2-(1H-[3-¹³C]indol-3-yl[¹³C]methyl)-malonic acid (c): 1.370 g (3.93 mmol) of (b) were dissolved in an aqueous solution of 3 g of LiOH in 25 ml of H2O. The solution was stirred by refluxing at 180° C. for 12 h. Then the solution was cooled to room temperature and acidified with 2N HCl. Hereby it was taken care that the temperature did not exceed 25° C. The solution was extracted twice with each 20 ml of ethyl acetate, dried (Na2SO₄) and the solvent was removed under reduced pressure at 40° C. Yield: 951 mg (83%).

4. 2-acetylamino-2-(1H-[3-¹³C]indol-3-yl[¹³C]methyl)-acetic acid (d): 930 mg of c were suspended in 20 ml of H₂O and the mixture was stirred at 100° C. for 1 h. After the mixture was cooled to room temperature and extracted three times with each 20 ml of ethyl acetate. The organic layers were dried with Na₂SO₄ and the solvent was removed under reduced pressure at 40° C. Yield: 540 mg (79%).

5. 2-amino-2-(1H-[3-¹³C]indol-3-yl)[3′-¹³C]propionic acid (D,L-[3,3′-¹³C₂]tryptophan): 520 mg of (d) were stirred at 150° C. in 2.0 ml of hydrazine in a sealed pressure tube for 15 h. After that the hydrazine was evaporated under reduced pressure. The residue was dissolved in 5 ml of H₂O and purified on an ion exchanger (Dowex 50 W X8) by elution with aqueous ammonia (25%). Yield: 195 mg (45%).−M=206.21; MS: m/z (%): 206 (3), 161 (2), 132 (100), 118 (5), 112 (26), 97 (29), 56 (31). ¹H-NMR (600 MHz, DMSO): d=3.01 (dm, ¹^{_{J}}CH=127.2 Hz, 1 H), 3.31 (dm, ¹^{_{J}}CH=129.3 Hz, 1 H), 3.50 (m, 1H), 6.96 (dd, ³^{_{J}}HH=7.5 Hz, 7.5 Hz, 1 H), 7.05 (dd, ³^{_{J}}HH=7.5 Hz, 7.5 Hz, 1 H), 7.24 (d, ³^{_{J}}HH=6.6 Hz, 1 H), 7.35 (d, ³^{_{J}}HH =7.8 Hz, 1 H), 7.56 (d, ³^{_{J}}CH=7.8 Hz, 1 H), 7.64 (s, 2 H, NH₂), 11,05 (s, 1 H, NH). ¹³C-NMR (151 MHz, DMSO): d=27.17 (d, ¹^{_{J}}CC =49.0 Hz), 54.75 (d, ¹^{_{J}}CC=49.0 Hz), 109.54 (d, ¹^{_{J}}CC=49.0 Hz), 111.33, 118.20, 118.36, 120.80, 124.14 (d, ¹^{_{J}}CC=53.9 Hz), 127.30 (d, ¹^{_{J}}CC=53.9 Hz), 136.34, 170.51.

Tissue Culture of Developing Maize Kernels

Plants of the commerical hybrid Pioneer 3394 were grown in a field at Johnston, Iowa. Ear shoots were kept bagged until plants were self pollinated, 2-3 days after silk emergence. Ears were harvested four days after pollination (DAP), placed on ice, and processed within four hours of harvesting essentially following the method of Gengenbach (1977). In the field, protruding silks were cut off and the most outer husks were removed. Once in the laboratory, all steps were conducted in a laminar flow hood using aseptic conditions. Holding ears by the shank, each ear was sprayed with alcohol and flamed before removing the outer husks. When only the most inner layer of husks was left, the ear was again lightly sprayed with alcohol and flamed. Sections of kernels, two kernels wide and with cob tissue at least 3 mm deep, were removed from the middle third of the ear. The wedges were further sectioned to eight-kernel blocks and the two kernels at each end of the cob piece was carefully removed to leave four developing kernels per block. Care was taken during excision to prevent drying out of kernels and cob tissue. The culture trays consisted of disposable Phytatrays (Sigma P5929, St. Louis, Mo.) each with a sterile 1-1.5 cm deep brick of florist foam (Wet Foam Block, StyroFab, Waxahachie, Tex.) were used. The tray lid (used as base) was filled with 50-80 ml of culture medium, enough to completely soak the foam and leave 2 ml of free medium. Using florist foam as substrate only 1-2 ml of medium were used per harvested kernel. Culley et al (1984) plating on agar media, used an average of 8 ml of medium per kernel; Singletary et al. (1989) using liquid medium and a wire/paper platform, employed 23-31 ml of medium per kernel.

Kernel blocks were pressed onto the wet foam brick until the kernels were leveled with the surface of the brick. On average, each tray contained twelve four-kernel blocks. With a hot dissecting needle, four small holes were made high on the sides of the tray lids to allow for some gas exchange. Foam bricks and liquid culture medium were sterilized by autoclaving for 30 min. The culture medium used was a modification of that described by Gengenbach and Jones (1994) and contained 80 g of sucrose or, in case of the [U-¹³C₆]glucose labeling experiment, 80 g of glucose, 4.33 g of Murashige Skoog Salts, 2 g of L-asparagine monohydrate, 400 μg thiamine hydrochloride, and 10 mg streptomycin sulfate per liter. The medium was adjusted to pH 5.8 with 4N Hcl before and autoclaved for 35 min. Cultures were incubated at 24° C., in the dark, and inspected every three days for possible contamination. After 7 days in culture, kernel blocks were transferred to fresh culture trays containing labeled medium and incubated for an additional 19 days with no changes of medium. Overall, kernels were harvested 30 days after pollination (DAP) and frozen at −80° C.

Isolation of IAA and Amino Acids.

Frozen kernels were grinded in liquid nitrogen, then extracted 3 times with 70% acetone in H₂O and twice with n-hexane/acetone, 1:1 (v/v). The residue was divided in half for tryptophan isolation and isolation of the other amino, acids. For purification of IAA the volume of the supernatant was reduced to 30%. The solution was adjusted to pH 14 by addition of 10N NaOH and incubated for 1 h to allow hydrolysis of conjugates (Ueda and Bandurski, 1969), then adjusted to pH 2 by addition of concentrated HCl and extracted 3 times with 0.7 volumes of ethyl acetate/n-hexane (3:1, v/v). The solvent was removed and the residue was dissolved in 2 ml of MeOH. IAA was purified by 2 sequential isocratic HPLC steps using a Merck nucleosil RP18 column (10×250 mm; flow rate, 6 ml/min). The effluent was monitored photometrically (278 nm). For the first HPLC step MeOH/HOAc/H₂O (10:9:81, v/v) was used as solvent. IAA eluted at 25 min. For the second HPLC step MeOH/HOAc/H₂O (40:5:54, v/v) was used as solvent. IAA eluted at 6 min. Fractions containing IAA were combined and extracted twice with ethyl acetate. The organic phase was evaporated to dryness under reduced pressure.

An aliquot of the solvent-extracted tissue was subjected to alkaline hydrolysis and tryptophan was isolated as described earlier (Eisenreich et al., 1991). A second aliquot of biomass was boiled for 24 h under reflux in 6M HCl containing 4% (v/v) thioglycolic acid. Amino acids were separated by ion exchange chromatography as described (Eisenreich et al., 1991). Leucine and tyrosine were further purified by reversed phase HPLC using a Macherey & Nagel RP18 column (21×250 mm) with water as eluent. The retention volumes were 120 and 150 ml, respectively. Histidine was purified by preparative TLC on silica plates that were developed with n-butanol/acetic acid/H₂O, 4/1/1 (v/v). Histidine was detected by spraying a small section of the plate with ninhydrin.

GC/MS Analysis.

Electron impact mass spectroscopy (EI MS), high resolution EI MS and gas chromatography/mass spectrometry (GC/MS) were performed on a Finnigan MAT 90 double focussing mass spectrometer, equipped with an EI ion source operated at 70 eV. In order to derivatize IAA to its methylester, 0.1 mg of IAA were dissolved in 100 μl of methanol, and 20 μl of trimethylchlorosilane were added. After 2 h at 25° C. the solvent was blown off with nitrogen. For sample injection the residue was dissolved in 100 μl of methanol. Trp was derivatized to Tris-(trimethylsilyl)-tryptophan: 50 μl MSTFA were added to 0.1 mg of Trp. The mixture was kept at 25° C. for 2 h and then injected on the GC column. For GC/MS a Varian GC 3400 gas chromatograph with a fuised silica DB-5ms capillary column (30 m ¥0.25 mm, coated with a 0.1 μm layer of liquid phase) and helium as carrier gas was used for sample separation. The injector temperature was kept at 300° C., injection volumes were 0.2-0.4 μl of a 1-2% (w/v) solution. Temperature program: 2 min isothermal at 50° C., then 10° C./min up to 300° C., finally 15 min isothermal at 300° C.

NMR Spectroscopy.

¹H and 13C NMR spectra of isolated metabolites were recorded at 500.13 MHz and 125.76 MHz, respectively, with a Bruker DRX 500 spectrometer equipped with a dual ¹H/¹³C probehead. Synthetic compounds were analyzed using Bruker AMX 600 and ARX 300 spectrometers. ¹³C NMR spectra were measured as follows: 45° pulse (3 μsec); repetition time, 3.2 sec; spectral width, 29 kHz; data set, 64 kilo-words; temperature, 10°; zero-filling to 128 kilo-words, and gaussian apodization prior to Fourier transformation; ¹H decoupling by WALTZ16 during acquisition and relaxation. The IAA signal assignments are based on two-dimensional homo- and heteronuclear NMR analysis (DQF-COSY, HMQC, HMBC, data not shown). IAA was dissolved in deuterated methanol. Isolated tryptophan and tyrosine were dissolved in 0.1M NaOD. Leucine and histidine were dissolved in 0.1M DCl.

Analysis of ¹³C Enrichment.

For each amino acid analyzed the ¹³C enrichment of one carbon carrying a proton atom was calculated after integration of ¹³C induced satellite signals in the ¹H spectrum. This ¹³C enrichment was set as standard to calculate the ¹³C enrichment of the other positions in the ¹³C spectrum after calibration with a natural abundance sample. The mean ¹³C enrichment of compounds isolated form kernels labeled with [U-¹³C₆]glucose was calculated from the ¹³C enrichment of all proton carrying positions.

Analysis of Isotopomer Composition.

In a ¹H decoupled ¹³C NMR spectrum the incorporation of one ¹³C atom results in a singlet signal. The presence of a second neighboring ¹³C nucleus results in a doublet signal with a coupling constant of 30-70 Hz. If two neighboring ¹³C nuclei, characterized by different coupling constants, are incorporated a doublet of a doublet is observed. If the coupling constants are the same, a 1:2:1 signal is observed. For each carbon position the central singlet signal, the doublet satellite signals and the double doublet satellite signals were integrated and the fraction of the total integral was calculated. This fraction represents the fraction of multiply ¹³C labeled isotopomers of all ¹³C containing isotopomers (Table 1, D). This value multiplied with the ¹³C enrichment (e) yields the isotopomer content (c) in mol % (Table 1, C).

Calculations for the Analysis of the [U-¹³C₆]glucose Labeling Experiment.

To allow comparison of ¹³C NMR data of metabolites with different rate of de novo biosynthesis the data were normalized to 100% de novo biosynthesis. The normalized fraction of multiply ¹³C labeled isotopomer n, which we introduce here (Table 1, E) represents the fraction of the multiply ¹³C labeled isotopomer of all ¹³C containing isotopomers that result from the incorporation of ¹³C nuclei that derive from [U-¹³C₆]glucose. It can be calculated from the isotopomer content (c) and the ¹³C abundance (e):

n=c (e−1.1%) as the natural abundance of ¹³C is 1.1%.

TABLE 1
Incorporation of deuterium from L-[²H₅]Trp into IAA and Trp. IAA and
tryptophan isolated from kernels labeled with 20
mM L-[²H₅]Trp (A), 2 mM L-[²H₅]Trp
(B), 2 mM L-[²H₅]Trp and 2 mM unlabelled
indole as competitor (C) were analyzed by GC/MS.
For Trp the relative mass distribution of the N-sylilated quinolinium
ion is shown (m₂₀₂ + m₂₀₇ = 100%). For IAA the relative mass
distribution of the quinolinium ion (m_{189 +}
m₁₉₃ + m₁₉₄ = 100%) and the molecular ion
(m₁₈₉ + m₁₉₃ + m₁₉₄ = 100%) is shown. The
identity of [ring-¹H₅]IAA (m = 130) [ring-²H₄]IAA (m = 134)
and [ring-²H₅]IAA (m = 135) was confirmed by HR-EI MS.

| | Ion | A | B | C |
|---|---|---|---|---|
| Trp | 202 | 36% | 53% | 53% |
| 207 | 64% | 47% | 47% | |
| IAA | 130 | 11% | 76% | 61% |
| 134 | 42% | 21% | 29% | |
| 135 | 47% | 3% | 10% | |
| 134 + 135 | 89% | 24% | 39% | |
| 189 | 11% | 75% | 62% | |
| 193 | 41% | 20% | 23% | |
| 194 | 48% | 5% | 15% | |
| 193 + 194 | 89% | 25% | 38% | |

### RESULTS

Incorporation of L-[ring-²H₅]tryptophan into IAA and Proteinogenic Tryptophan.

The standard protocol of maize kernel culture was modified in order to make more efficient use of labeled precursor. These changes allowed to use only 1-2 ml of medium per kernel. No change in kernel growth was visible. It was tested whether developing maize kernels incorporate label from Trp into IAA. Kernels were grown for 19 days on standard medium containing 20 mM L-[ring-²H₅]Trp (A) or 2 mM L-[ring-²H₅]Trp (B). In a third experiment (C) kernels were incubated in medium containing 2 mM L-[ring-²H₅]Trp and 2 mM unlabelled indole, to test whether indole is an efficient competitor for Trp as IAA precursor.

IAA was isolated from each sample and the incorporation of ²H from L-[²H₅]Trp was monitored by GC/MS. In addition Trp was isolated from total protein and analyzed by GC/MS to monitor the uptake of the amino acid Trp into the tissue. The mass distribution of the quinoline ion region of the different IAA and Trp samples is given in Table 1. In all samples derived from kernels labeled with L-[²H₅]Trp a high abundance of the m+5 peak was observed. To confirm that the m+5 ion derives from the incorporation of 5 ²H atoms, aliquots of the IAA (A) sample were subjected to HR-EI MS. For the fully protonated quinoline ion the mass of 130.06537 was observed (calculation 130.06567). For the [²H₅]quinoline ion the mass of 135.09705 was observed (calculation: 135.09706). In addition, the abundant [²H₄]quinoline (m+4) ions, probably derived from the partial exchange of ²H during the purification of [²H₅]IAA, were observed in labeled IAA samples (calculated: m=134.0908, observed: m=134.0903). The same isotope shift was observed for the m/z 189 IAA molecular ion. From the quinolinium isotope distribution the relative enrichment was determined: IAA(A): 89%, IAA(B): 25%, IAA(C): 39%, Trp(A): 64%, Trp (B): 47%, Trp (C): 47%. The incorporation of ²H from L-[²H₅]tryptophan into IAA indicated that the indole ring of Trp is efficiently incorporated into IAA. A reduction of this incorporation rate by indole was not observed.

Incorporation of [3,3′-¹³C₂]tryptophan into IAA and Proteinogenic Tryptophan

The next question was whether the side chain of IAA derives also from Trp or if IAA is synthesized via a speculative pathway that involves the breakage of the Trp 3,3′ bond. Kernels were labeled with 1 mM D,L-[3,3′-¹³C₂]tryptophan. IAA and Trp were isolated from this material and analyzed by ¹³C NMR spectroscopy. Satellite signals caused by ¹³C¹³C couplings were observed for IAA (C-3 and C-2′; J_{CC}=50.2 Hz) and Trp (C-3 and C-3′; J_{CC}=49.3 Hz). The relative signal intensities were compared to a natural abundance sample and a concentration of 8.6 mol % [3,2′-¹³C₂]IAA and 5.4 mol % [3,3′-¹³C₂]Trp was calculated. No significant enrichment of the [3-¹³C₁]IAA isotopomer was observed. These results show clearly that IAA is synthesized from Trp without breakage of the 3,3′ bond.

Investigating IAA Biosynthesis by Quantitative Assessment of Carbon Flux in Developing Maize Kernels. Studies With [U-¹³C₆]glucose and [1,2-¹³C₂]acetate.

Experiments with L-[ring-²H₅]Trp and D,L-[3,3′-¹³C₂]Trp showed that the aromatic amino acid tryptophan serves as an efficient precursor of IAA in developing maize kernels. However this result can not exclude alternative routes to IAA in this tissue. In order to assess the entire metabolic network leading to IAA we performed experiments with the unspecific precursors [U-¹³C₆]glucose and [1,2-¹³C₂]acetate followed by retrobiosynthetic NMR analysis. This technique allows a quantitative prediction of the general carbon flux in the biological system under study by a retrodictive analysis of ¹³C labeling patterns (for review see Bacher et al. 1999. For this purpose developing maize kernels were cultured on sterile medium supplied with 2 g [U-¹³C₆]or 3 g [1,2-¹³C₂]acetate per liter medium. After 19 days IAA, Trp, Leu, Tyr and His were isolated and were analyzed by ¹³C NMR spectroscopy. Isotopomers containing contiguous ¹³C atoms were quantified by analysis of ¹³C¹³C couplings. Isotopomers with two contigous ¹³C atoms result in a doublet signal due to ¹³C¹³C coupling. Isotopomers with three adjacent ¹³C atoms result in a double doublet reflecting two ¹³C couplings. From the relative signal intensities the abundances of the isotopomers [1′,2′-¹³C₂]Trp (w1), [2′,3′-¹³C₂]Trp (w2) and [1′,2′,3′-¹³C₃]Trp (w3) can be calculated (Table 2). The comparison of the isotopomer composition of the metabolite of interest (IAA) with the isotopomer composition (Table 2) of several primary metabolites allows to judge which metabolites are plausible precursors of IAA. Besides the biosynthesis from Trp (1) three alternative IAA biosynthetic pathways were tested for their quantitative relevance for IAA biosynthesis in vivo: The biosynthesis directly from IGP (4), the biosynthesis from indole and a C2 precursor that derives from acetyl-CoA (2) and the biosynthesis from indole and a C3 unit that derives from PEP or a triose phosphate (3). To compare the predicted and observed isotopomer distribution for these proposed biosynthetic pathways it was necessary to get information about the isotopomer composition of the following metabolites: IAA, Trp, ribose phosphate, indole, anthranilate, acetyl-CoA and PEP.

The isotopomer composition of Trp, Leu, Tyr and His was determined (Table 2). Based on established mechanisms of amino acid biosynthesis, the isotopomer distribution of central metabolites were reconstructed from the labeling data of the amino acids. Only a fraction of a given metabolite is synthesized de novo during the labeling period. To account for this fact the amount of each isotopomer was normalized to 100% de novo biosynthesis for each metabolite considered (see materials and methods). Specifically the abundance of the [1,2-¹³C₂]acetate isotopomer could be inferred from the normalized abundance of [1,2-¹³C₂]Leu as the acetate moiety of acetyl-CoA is incorporated into C-1/C-2 of leucine (Oaks, 1965). The abundance of [2,3-¹³C₂]PEP and [1,2,3-¹³C₃]PEP was derived from the abundance of [2,3-¹³C₂]Tyr (yl) and [1,2,3-¹³C₃]Tyr (y2). As indole is the direct precursor of Trp the abundance of [2,3-¹³C₂]indole and [3a,7a-¹³C₂]indole could be inferred from the abundance of [2,3-¹³C₂]Trp (w4) and [3a,7a-¹³C₂]Trp (w5), respectively. Anthranilic acid is a precursor of indole (for review see Schmid and Amrhein, 1995). [1,2-¹³C₂]anthranilic acid could be inferred from [3a,7a-¹³C₂]indole the precursor of [3a,7a-¹³C2]Trp (w5) (see FIG. 5 +L). Histidine derives from phosphoribosyl pyrophosphate (Wiater et al, 1972) and therefore the ribose labeling pattern can be determined from the histidine labeling data: [1,2-¹³C₂]ribose phosphate from [4,5- ¹³C₂]His (h1) and [3,4,5-¹³C₃]ribose phosphate from [1,2,3-¹³C₃]His(h2).

All metabolites isolated from kernels labeled with [U-¹³C₆]glucose showed incorporation of label (Table 2). From the absolute ¹³C enrichment of each metabolite the rate of de novo biosynthesis during the labeling period could be calculated. Using this enrichment data the abundance of multiple labeled isotopomers could be normalized (see materials and methods of this Example). In all metabolites analyzed we observed intactly incorporated blocks that resulted in ¹³C₂- and ¹³C₃-isotopomers in a much larger amount than expected from statistic incorporation of ¹³C. Normalized abundances of 0.39 for [1′,2′-¹³C₂]IAA, 0.70 for [2,3-¹³C₂]IAA and 1.05 for [3a,7a-¹³C₂]IAA were observed (Table 2). The relative abundance of the analyzed isotopomers (Table 2), normalized for 100% de novo biosynthesis, and the isotopomer compositions of the inferred metabolic intermediates were compiled. Using the isotopomer composition of these metabolic intermediates the labeling pattern of IAA via hypothetical mechanisms could be predicted. More specifically, the normalized abundance of [1′,2′-¹³C₂]IAA, [2,3-¹³C₂]IAA and [3a,7a-¹³C₂]IAA was predicted for the a Trp dependent (1) and 3 hypothetical Trp independent (2-4) pathways described above. The measured IAA data and the four predictions showed similar normalized abundance of the [2,3-¹³C₂]IAA and [3a,7a-³C₂]IAA (0.95 for all predictions shown) isotopomer. The ratio [2,3-¹³C₂]IAA [3a,7a-³C₂]IAA could be more accurately determined as it is not influenced by the rate of de novo biosynthesis. In this case the prediction 1, 2, 3 (0.65) and 4 (0.64) match well with the observed ratio (0.67) which indicates that comparable isotopomer composition of the indole ring can be reconstructed using IAA, Trp or Trp plus His primary data. This confirms the origin of IAA from the shikimic acid pathway and it shows that despite the complexity of the system the technique of retrobiosynthetic analysis can be applied. On the other hand the normalized abundance of the [1′,2′-¹³C₂]IAA isotopomer for the acetic acid side chain (Table 3), and the [1′,2′-¹³C₂]IAA/[2,3-¹³C₂]IAA ratio (Table 3) show significant differences for the observed and predicted isotopomer ratios for Trp as a precursor of IAA. These differences can be explained by separate Trp pools for IAA and Trp incorporated into proteins (see discussion below). These pools would differ in the biosynthetic origin of the serine side chain of Trp. The observed isotopomer ratios clearly exclude, however, that IAA is synthesized from IGP or from indole plus a metabolite derived from acetyl CoA or PEP. As comparable isotopomer compositions can be expected for PEP and triose phosphate, also a triose phosphate can be excluded as precursor of the side chain of IAA.

Kernels were also labeled with [1,2-¹³C₂]acetate. IAA and Trp isolated from this tissue did not show any significant enrichment of the [1′,2′-¹³C₂]IAA, [2′,3′-¹³C₂]Trp, [2,3-¹³C₂]IAA and [2,3-¹³C₂]Trp isotopomer. On the other hand 0.87 mol % [1,2-¹³C₂]Leu that derives from [1,2-¹³C₂]acetyl CoA was observed. This gives additional evidence that a hypothetical IAA biosynthetic pathway via indole plus a C2 unit derived from acetyl CoA does not play a major role for IAA biosynthesis in vivo.

TABLE 2
Isotopomer analysis of multiple ¹³C labeled IAA and proteinogenic
amino acids isolated from developing maize kernels
supplied with [U-¹³C₆]glucose:

| A | | B | C | D | E |
|---|---|---|---|---|---|
| IAA | 1′,2′-¹³C₂ | 1 | 0.54 | 0.22 | 0.39 |
| | 2,3-¹³C₂ | 2 | 1.00 | 0.40 | 0.70 |
| | 3a,7a-¹³C₂ | 3 | 1.48 | 0.59 | 1.05 |
| Trp | 1′,2′-¹³C₂ | w1 | 0.30 | 0.14 | 0.29 |
| | 2′,3′-¹³C₂ | w2 | 0.13 | 0.06 | 0.12 |
| | 1′,2′,3′-¹³C₃ | w3 | 0.36 | 0.17 | 0.35 |
| | 2,3-¹³C₂ | w4 | 0.64 | 0.30 | 0.62 |
| | 3a,7a-¹³C₂ | w5 | 0.98 | 0.64 | 0.95 |
| His | 4,5-¹³C₂ | h1 | 0.90 | 0.35 | 0.61 |
| | 1,2,3-¹³C₃ | h2 | 1.00 | 0.39 | 0.68 |
| Leu | 1,2-¹³C₂ | l1 | 1.01 | 0.47 | 0.97 |
| Tyr | 1,2-¹³C₂ | y1 | 0.32 | 0.12 | 0.21 |
| | 1,2,3-¹³C₃ | y2 | 1.05 | 0.40 | 0.68 |

A: isotopomers analyzed.
B: abbreviation for the analyzed isotopomer
C: mol % of isotopomer carrying ¹³C nuclei at least at the designated positions.
D: Fraction of multiple ¹³C labeled isotopomers of all isotopomers carrying ¹³C at the designated positions (see materials and methods).
E: Fraction of multiple ¹³C labeled isotopomers of all isotopomers carrying ¹³C at the designated positions incorporated during the labeling (see materials and methods). This normalization enables comparison of metabolites with different de novo biosynthetic rate using ¹³C enrichment data: IAA: 2.50% ¹³C, Trp: 2.14% ¹³C, Leu: 2.14% ¹³C, Tyr: 2.63% ¹³C, His: 2.57% ¹³C.

TABLE 3
Observed and predicted labeling pattern of IAA: Predictions are made on
the basis of metabolites derived from kernels labeled with [U-13C6]
glucose. The normalized abundance of [1′,2′-¹³C₂]IAA (1) and
[2,3-¹³C₂]IAA (2) and the ratio
[1′,2′-¹³C₂]IAA/[2,3-¹³C₂]IAA is given.

| | [1 ′,2′-¹³C₂]IAA | [2,3-¹³C₂]IAA | ratio |
|---|---|---|---|
| Trp | 0.47 | 0.62 | 0.76 |
| JGP | 0.68 | 0.61 | 1.11 |
| indole plus acetate | 0.97 | 0.62 | 1.56 |
| derived metabolite | | | |
| indole plus PEP derived | 0.89 | 0.62 | 1.43 |
| metabolite | | | |
| observed | 0.39 | 0.70 | 0.56 |

Four hypothetical IAA biosynthetic pathways were analyzed for their plausibility.
1.: Biosynthesis from Trp: [1′,2′-¹³C₂]IAA derives from [1′,2′,3′-¹³C₃]Trp (w3) plus [2′,3′-¹³C₂]Trp (w2).
2.: Biosynthesis from IGP: [1′,2′-¹³C₂]IAA derives from [4,5-¹³C₂]Iribose phosphate.
3.: Biosynthesis from indole plus an acetate derived metabolite: [1 ′,2′-¹³C₂]IAA derives from [1,2-¹³C₂]acetyl-CoA.
4.: Biosynthesis from indole plus a PEP or triose phosphate derived metabolite: [1 ′,2′-¹³C₂]IAA derives from [1,2,3-¹³C₃]PEP plus [2,3-¹³C₂]PEP. For a biosynthesis from IGP [2,3-¹³C₂]IAA derives from [1,2,3-¹³C₃]ribose phosphate, for the other hypothetical pathways [2,3-¹³C₂]IAA derives from [2,3-¹³C₂]indole.

Discussion

Plants can synthesize IAA by multiple pathways. Several Trp-dependent pathways have been proposed. IAA synthesis can also occur independent of Trp via a Trp precursor, e.g. indole or indole-3-glycerol phosphate. This biosynthetic redundancy (reviewed in Bartel 1997; Normanly and Bartel 1999) has complicated the elucidation of IAA biosynthesis. Different pathways can exist in different tissues or in different developmental stages of one plant.

Relatively large amounts of IAA conjugates accumulate during development in the maize endosperm (Bandurski et al., 1999). IAA from this deposit is probably required for polar embryo development and for development of the maize seedling. Seedlings that are not dissected from the kernel import IAA from the endosperm (Epstein et al., 1980). IAA de novo biosynthesis is initiated late in seedling development (Jensen and Bandurski, 1996) and is not detectable in the first week after germination. Therefore biosynthesis, conjugation and mobilization of IAA can be studied in the maize kernel. The aim of our study was to elucidate IAA biosynthesis in this model system under physiological conditions.

Label from Trp is efficiently incorporated into IAA in the maize endosperm

Crude enzyme preparations from maize endosperm converted both indole (Rekoslayskaya and Bandurski, 1994; Rekoslayskaya 1995) and Trp (Östin et al., 1999) into IAA. We modified the kernel tissue culture system in order to obtain efficient labeling of IAA in a system that closely resembles the in vivo situation (Cobb and Hannah, 1983; Cully et al., 1984). Deuterated and ¹³C labeled Trp were used for the incorporation experiments. When 2 mM L-[²H₅]Trp was used, the relative enrichment was 24% for IAA and 47% for Trp isolated from total protein. In contrast, with 20 mM L-[²H₅]Trp 89% of IAA and 64% of Trp were labeled. These differences in specific enrichment could indicate that two pools for Trp exist in the system and that these pools are not equally well accessible for external Trp. Alternatively or in addition, a significantly higher de novo biosynthesis rate of IAA in the presence of 20 mM Trp could account for these results. Unlabelled indole did not suppress the conversion of Trp to IAA. These findings suggest that Trp-dependent IAA biosynthesis is the predominant route in the developing maize endosperm. Similar results were obtained with liquid endosperm preparations (Östin et al., 1999).

NMR analysis of IAA that incorporated label from D,L-[¹³C₂]Trp clearly showed that IAA is synthesized without breakage of the 3,3′ bond. This excludes a hypothetical pathway that would involve the breakdown of Trp to yield indole as an intermediate of IAA biosynthesis.

Retrobiosynthetic analysis of IAA biosynthesis.

The Trp labeling experiment in the in vitro culture of kernels provided evidence for Trp-dependent IAA biosynthesis. However, it can not be excluded that specific precursors can influence the biosynthetic route. We therefore evaluated the origin of the IAA carbon skeleton when specific precursors were omitted. The high concentration of IAA in the maize endosperm and the improvement of the kernel culture described in this manuscript, allowed to isolate IAA in quantities sufficient for retrobiosynthetic analysis based on ¹³C NMR spectroscopy. Uniformly labeled glucose as a general precursor provided an assessment of the IAA biosynthetic route reflecting the natural situation. The results obtained, exclude IAA synthesis from indole-3-glycerol phosphate because the abundance of the [1′,2′-¹³C₂]IAA isotopomer is by far too low to originate from [2′,3′-¹³C₂]IGP and [1′,2′,3′-¹³C₃]IGP. According to this low abundance of [1′,2′-¹³C₂]IAA also an IAA formation from indole and condensation with metabolites that derive from the PEP pool or from acetyl-CoA can be excluded (Table 3). The exclusion of acetyl-CoA and acetate derived metabolites like glycolate, mevalonate, etc. as precursors of the side chain of IAA was confirmed by feeding with [1,2-¹³C₂]acetate. No incorporation of acetate into the IAA side chain was observed, although acetate was efficiently metabolized, e.g. to lysine.

However the conversion of Trp to IAA can also be not directly deduced, since the isotopomer analysis of the Trp and IAA side chains provided not identical values (Table 2,3). In contrast the labeling patterns of the indole moiety of Trp and IAA are in good agreement (Table 3). This result could indicate that a fraction of IAA is synthesized from a Trp precursor, which remains purely speculative since the above mentioned metabolites for the acetic acid side chain can be excluded.

Alternatively two different Trp pools have to be postulated. This hypothesis would predict that the side chains of Trp in the two pools would have different isotopomer pattern. The side chain of Trp is derived from serine. There are two different serine pathways in plants: a pathway via glycine and a pathway via 3-phosphoglycerate (Ho et al., 1998, Ho et al., 1999). The expected abundance of the [1′,2′,3′-¹³C₃]Trp isolated form [U-¹³C₆]glucose labeled kernels should differ dramatically for these two serine biosynthetic pathways: When serine is synthesized from two molecules of glycine, no [1′,2′,3′-¹³C₃]Trp should be detectable. In contrast, when serine is synthesized from 3-phosphoglycerate that derives from PEP, the normalized abundance of [1′,2′,3′-¹³C₃]Trp should be similar to [1,2,3-¹³C₃]PEP. We observe a normalized abundance of 0.35 for [1′,2′,3′-¹³C₃]Trp and 0.68 for [1′,2′,3′-¹³C₃]PEP, deduced from [1′,2′,3′-¹³C₃]Tyr (Table 2). From these data we conclude that the serine side chain of Trp that is incorporated into proteins is synthesized via both pathways that contribute approximately equal amounts to this serine pool. Similar results have been obtained for serine biosynthesis in heterotrophic Beta vulgaris cell cultures (Werner, 1996). The serine side chain of the Trp, from which IAA is synthesized, should derive to a larger extent from two molecules of glycine. This explanation and the Trp precursor feeding experiments indicate separate Trp pools for IAA and protein biosynthesis.

In summary we conclude from our data that in developing maize kernels IAA is synthesized from tryptophan. There is no evidence for a major contribution of a Trp independent pathway. Several pathways for the conversion of Trp to IAA have been proposed. A few genes have been cloned (reviewed in Normanly and Bartel, 1999) that encode candidate genes of these pathways. These molecular “probes” can now be used to further dissect IAA metabolism in the maize endosperm.

### LITERATURE CITED

Bacher A, Rieder C, Eichinger D, Arigoni D, Fuchs G, Eisenreich W (1999) Elucidation of novel biosynthetic pathways and metabolite flux patterns by retrobiosynthetic analysis. FEMS Microbiol Rev 22: 567-589

Bandurski RS, Cohen JD, Slovin J, Reinecke DM (1995) Auxin biosynthesis and metabolism. In JP Davies, ed, Plant Hormones: Physiology, Biochemistry and Molecular Biology, Kluver Academic Publishers, Dordrecht., pp 39-65

Bandurski RS, Kowalczyk S, Lezniki A, Mekhedov S, Momonoki Y, Oguri S (1998) Metabolic targets for control of IAA levels in maize. Plant Growth Regulation Society of America Proceedings of the 25th Annual Meeting, 26: 181-186.

Bartel B, Fink GR (1994) Differential regulation of an auxin producing nitrilase gene family in Arabidopsis thaliana. Proc Natl Acad Sci USA 91: 6649-6653

Bartel B (1997) Auxin biosynthesis. Ann Rev Plant Physiol Plant Mol Biol 48: 51-66

Bartling D, SeedorfM, Schmidt RC, Weiler EW (1994) Molecular characterization of two cloned nitrilases from Arabidopsis thaliana; key enzymes in the biosynthesis of the plant hormone indole-3-acetic acid. Proc Natl Acad Sci USA 91: 6021-6025

Bialek K, Michalczuk L, Cohen JD (1992) Auxin biosynthesis during seed germination in Phaseolus vulgaris. Plant Physiol 100: 509-517

Cobb BG, Hannah LC (1983). Development of wild type, shrunken-1 and shrunken-2 maize kernels grown in vitro. Theor Appl Genet 65: 47-51

Cully DE, Gengenbach BG, Smith JA, Rubenstein I, Connelly JA, Park W (1984) Endosperm protein synthesis and L-[³⁵S]methionine incorporation in maize kernels cultured in vitro. Plant Physiol 74: 38-394

Eisenreich W, Schwarzkopf B, Bacher A (1991) Biosynthesis of nucleotides, flavins and deazaflavins in Methanobacterium thermoautotrophicum. J Biol Chem 266: 9622-9631

Epstein E, Cohen JD, Bandurski RS (1980) Concentration and metabolic turnover of indoles in germinating kernels of Zea mays. Plant Physiol 65: 415-421

Gengenbach BG (1977a) Development of maize caryopses resulting from in-vitro pollination. Planta 134: 91-93

Gengenbach BG. and Jones RJ (1994) In vitro culture of maize kernels. In M Freeling, V Walbot, eds, The Maize Handbook, Springer Verlag, pp 705-708

Hillebrand H, Bartling D, Weiler E (1998) Structural analysis of the nit2/nit1/nit3 gene cluster encoding nitrilases, enzymes catalyzing the terminal activation step in indole-acetic acid biosynthesis in Arabidopsis thaliana. Plant Mol Biol 36: 89-99

Ho CL, Noji M, Saito M, Yamazaki M, Saito K (1998) Molecular characterization of plastidic phosphoserine aminotransferase in serine biosynthesis from Arabidopsis. Plant J 16: 443-52

Ho CL, Noji M, Saito K (1999) Plastidic pathway of serine biosynthesis. Molecular cloning and expression of 3-phosphoserine phosphatase from Arabidopsis thaliana. J Biol Chem 274: 11007-11012

Jensen PJ, Bandurski RS (1996) Incorporation of deuterium into indole-3-acetic acid and tryptophan in Zea mays seedlings grown on 30% deuterium oxide. J Plant Physiol 147: 697-702

Michalczuk L, Ribnicky DM, Cooke TJ, Cohen JD (1992) Regulation of indole-3-acetic acid biosynthetic pathways in carrot cell cultures. Plant Physiol 100: 1346-1353.

Normanly J, Cohen JD, Fink GR (1993) Arabidopsis thaliana auxotrophs reveal a tryptophan-independent biosynthetic pathway for indole-3-acetic acid. Proc Natl Acad Sci USA 93: 10355-10359

Normanly J, Bartel B (1999) Redundancy as a way of life—IAA metabolism. Curr Opin Plant Biol 2: 207-213

Oaks A (1965) The synthesis of leucine in maize embryos. Biochim Biophys Acta 11: 79-89

Östin A, Ilic N and Cohen JD (1999) An in-vitro system from maize seedlings for tryptophan independent IAA biosynthesis. Plant Physiol 119: 173-178

Rapparini F, Cohen JD, Slovin J (1999) Indole-3-acetic acid biosynthesis in Lemna gibba studied using stable isotope labeled anthranilate and tryptophan. Plant Growth Reg, in press

Rekoslavskaya NI, Bandurski RS (1994) Indole as a precursor of indole-3-acetic acid in Zea mays. Phytochem 35: 905-909.

Rekoslavskaya NI (1995) Biosynthesis pathways of IAA and tryptophan in the developing maize endosperm: In vitro study. Fiziologiya Rastenii (Moscow) 42: 165-174

Schmid J and Amrhein N (1995) Molecular organizationof the shikimate pathway in higher plants. Phytochem 39: 737-749

Sekimoto H, Seo M, Kawakami N, Komano T, Desloire S, Liotenberg S, Marion-Poll A, Cabaoche M, Kamiya Y, Koshiba T (1998) Molecular cloning of aldehyde oxidases in Arabidopsis thaliana. Plant Cell Physiol 39: 433-442

Singletary GW, Below FE. (1989) Growth and composition of maize kernels cultured in vitro with varying supplies of carbon and nitrogen. Plant Physiol 89: 341-346

Singletary GW, Doehelert DC, Wilson, CM, Muhitch MJ, Below FE (1990) Response of enzymes and storage proteins of maize endosperm to nitrogen supply. Plant Physiol. 94: 858-864

Tam YY, Normanly J (1998) Determination of indole-3-pyruvic acid levels in Arabidopsis thaliana by gas chromatography selected ion monitoring mass spectroscopy. J Chromatography 800: 101-108

Thimann KV (1977) Hormone action in the whole life of plants. Univ. of Massachusetts Press, Amherst

Ueda M, Bandurski RS (1969) A quantitative estimation of alkali-labile indole-3-acetic acid compounds in dormant and germinating maize kernels. Plant. Physiol. 44: 1175-1181

Werner I (1996) Biosynthese von Gallussäure und Aminosäuren in Pflanzen. Thesis, Technische Universität, Munich

Wiater A, Krajewska-Grynkiewicz K, Klopotowski T (1971) Histidine biosynthesis and its regulation in higher plants. Acta Biochim Pol 18: 299-307

Wright AD, Sampson MB, Neuffer G, Michalczuk L, Slovin JP, Cohen JD (1991). Indole-3-acetic acid biosynthesis in the mutant maize orange pericarp, a tryptophan auxotroph. Science 254: 998-1000

What is claimed:

1. A method for kernel culture comprising:
a) harvesting ears;
b) using aseptic conditions, removing sections of kernels from the ears, said sections comprising from 1 to 5 kernels and at least about 3 mm of underlying cob;
c) sub-sectioning the sections of step (b) into sub-sections having a kernel-to-cob ratio of less than 1:6;
d) placing said sub-sections into sterile pieces of florist foam in a container with culture medium;
e) pressing the sub-sections into the florist foam pieces; and
f) culturing the sub-sections.
